(19) United States
(12) Patent Application Publication
(10) Pub. No.: US 2019/0274612 A1
(43) Pub. Date: Sep. 12, 2019

(54) System and Method for Real-time Reporting, Interacting, and Updating of Guardian Actions Within a School System

(71) Applicant: Pedro Chavez, JR., Orange, CA (US)

(72) Inventor: Pedro Chavez, JR., Orange, CA (US)

(21) Appl. No.: 15/917,574

(22) Filed: Mar. 10, 2018

(51) Int. Cl. A61B 5/16; G09B 5/14; G06Q 50/20; G06Q 50/26; G06F 9/54

(52) U.S. Cl. CPC A61B 5/167; G09B 5/14; G06Q 50/20; G06F 9/542; A61B 5/165; A61B 5/168; G06Q 50/265

(57) ABSTRACT

An interactive guardian communication system is disclosed. The interactive communication system dynamically connects guardians of students to teachers, administrators, students, and/or school support staff. Real-time data is provided to one or more electronic devices enabling dynamic individual and group communications between guardians, students, teachers, administrators, and/or school support staff. Identification and authentication of users within the interactive communication system enables secure data input, secure data access, and secure data output reporting.

### FIELD OF THE INVENTION

The present invention provides interactive communication systems and methods within an educational system.

### BACKGROUND

School systems suffer from challenges in effective communication between students, guardians, and school support staff. Bullying is a major problem in most schools and leads some parents to withdraw student from public school systems. Most bullying is a result of ineffective communication. Other problems including social, emotional, and physical health of students, parents, and school support staff could be recognized and treated with an effective school communication system in place. There is a need for a school communication system which enables proactive communication strategies allowing early detection, correction, mitigation, and treatment of social, emotional, and physical difficulties faced by students, guardians, and school support staff.

### SUMMARY

An interactive guardian communication system is disclosed. The interactive communication system dynamically connects guardians of students to teachers, administrators, students, and/or school support staff. Real-time data is provided to one or more electronic devices enabling dynamic individual and group communications between guardians, students, teachers, administrators, and/or school support staff. Identification and authentication of users within the interactive communication system enables secure data input, secure data access, and secure data output reporting.

A system that interacts with one or more guardians of one or more students includes: a first electronic device including a processor and memory non-transitively programmed to receive identification information of the one or more students in relation to event information, location information, and/or behavior information of the one or more students; a second electronic device including a processor and memory non-transitively programmed to receive the identification information related to the one or more students and the event information, the location information, and/or the behavior information, wherein the second electronic device determines, one or more actions to take based on the identification information, the event information, the location information and/or the behavior information of the one or more students; and a third electronic device including a processor and memory non-transitively programmed to receive the one or more determined actions and interactively notify the one or more guardians of the one or more students of a final determined action.

The third electronic device may be a smartphone, tablet, or computer system of the one or more guardians. The third electronic device may interactively notify the one or more guardians by requiring the one or more guardians to acknowledge the final determined action or approve the final determined action. The first electronic device may receive the identification information by scanning indicia on an identification card of the one or more students or by manual input of identification information of the one or more students. The final determined action may be a notice that the one or more students have one or more of missing assignments, late assignments, quizzes, presentations, projects, important homework due, assessments (school or state tests), or all assignments are turned in. The third electronic device may interactively notify the one or more guardians by requiring the one or more guardians to approve a tardy or absent child and the final notification is of the approved tardy or absence. The indicia may be a machine-readable image, a barcode, or an alphanumeric code. The event may be a financial charge to an account of the one or more students. The behavior may be a positive behavior or a negative behavior. The location information may be determined, in part, by GPS (global positioning system) within the first electronic device. The location information may be determined by a user input within the first device. The user of the first device may be one or more of a teacher, an administrator, support staff, campus security, volunteer, school designee, or a bus driver. The user of the first device may be authenticated by scanning an identification card of the user. The second electronic device may be a server, database server, private server, intranet server, remote computer, or cloud-based computing system. The third electronic device may interactively notify the one or more guardians by requiring the one or more guardians to choose from a selection of determined actions before the final determined action is presented to the one or more guardians. The third electronic device may display a historical log of locations to the one or more guardians of the one or more students. The historical log may include an indication of the one or more students being in a correct location at a correct time. The correct location may be a classroom, sporting event, field trip, school activity outside of school site, lunchroom, bus, or home. The one or more guardians may request a message to be delivered to the one or more students through interaction with the third electronic device. The message may be delivered to the one or more students when the student identification card is scanned by the first electronic device after the request by the one or more guardians.

A method for facilitating student compliance includes a processor and memory non-transitively programmed to: receive and accept student logins within a predetermined geographical network location, a predetermined network address, or a predetermined virtual private network; determine if the student logins are qualified student logins; form one or more groups of qualified student logins into qualified student groups; allow access to physical school resources and software resources based on predetermined privileges given to various qualified student logins and/or qualified student groups; and revoke specific school privileges or specific software access when the student is non-compliant with predetermined school rules.

The received and accepted student logins may be a result of scanning a barcode. The barcode may be a barcode printed on a student identification card of the student. The barcode may provide student network access on a student device after the student device scans the barcode. The scanning the barcode may provide a specific location of the student in relation to a classroom of the school. The scanning the barcode may be used to take attendance of the student. A school device or staff device may be used to scan the barcode. The physical school resources may be one or more of school activities, school meals, school networks, school rooms, school libraries, school sporting events, school snacks, school lockers, or school passes. The qualified student logins may be determined, in part, as a function of positive behavior or negative behavior of the student. The specific location may be determined, in part, by GPS (global positioning satellite) within a device of the student. The specific location may be determined, in part, by GPS (global positioning satellite) within a school device or a staff device. The school device or staff device may be a device of one or more of a teacher, an administrator, support staff, campus security, or a bus driver. The received and accepted student logins may be a result of manually inputting student identification information. The received and accepted student logins may be a result of facial recognition, voice recognition, retinal scanning, finger printing, or a biometric identifier. The method may notify a guardian or guardian of the student when the student is logged in or logs in. The notifying of the guardian may include a notification of the qualification status of the student of the guardian. The notifying of the guardian may be on a guardian device. The notifying of the guardian includes any necessary steps the student needs to take to become a qualified or compliant login. The predetermined school rules may be accessible on a student device and a guardian device. The predetermined school rules may be modifiable by school staff or school administrators and updated on the student device and the guardian device.

A method of data collecting and data sharing includes a computing system including a processor and memory non-transitively programmed to: receive data about one or more students from school officials, teachers, or guardians as an input to the computing system; update stored behavioral information about the one or more students based on the received data; track historical and real-time behavioral trends of the one or more students based on the received data; evaluate the real-time behavioral trends of the one or more students to determine a current behavioral state of the one or more students; evaluate the historical behavioral trends of the one or more students to determine a current behavioral contract state of the one or more students; and electronically share the current behavioral state or a current behavioral contract state of the one or more students with a school official, a guardian, or a teacher when the stored behavioral information is updated or modified.

The electronically share step may be accomplished by a text message, prerecorded phone message, email, and/or a push notification. The current behavioral contract state of the one or more students' may include restricted activities, behaviors, or clothing. The real-time behavioral trends of the one or more students may include positive behavior indicators and negative behavior indicators. The method may additionally comprise a predetermined negative behavior threshold. The guardian may be notified by the electronically share step when the predetermined negative behavior threshold is exceeded. The computing system may include wireless networks and wireless computing devices. The wireless computing devices may include one or more of iPad, iPods, tablets, smartphones, laptops, computers, wireless optical scanners, wireless data cards, and personal electronic devices. The method may include scanning an identification card of a school official, staff member, or teacher before the receive data about one or more students step. The current behavioral state of the one or more students may automatically trigger an automatic appointment with a school counselor or a school official. The automatic appointment may be sent to one or more of a guardian device, a teacher device, a student device, a school official device, or a counselor device. The method may further include scanning an identification card of a student before the receive data about one or more students step. The receive data step may include a guardian sharing confidential student information related to a real-time student behavior that may continue at school which started before school. The tracking may include visual indicators of behavioral student progress trends over hours, days, weeks, and months. The method may further comprise associating behavioral trends of a single student with a plurality of students. The method may further comprise notifying guardians when a student behavior is associated with a behavior of another student. The method may further comprise notifying guardians when a student behavior is associated with or linked to a behavior of a group of students. The method may further comprise a notification sent to a student device of the behavioral student progress trends over hours, days, weeks, and months. The student device may be a smartphone, laptop, computer, tablet, iPod, network enabled device, or iPad. The method may further comprise scanning a barcode before the receive data about one or more students step.

### BRIEF DESCRIPTION OF THE DRAWINGS

In order that the advantages of the invention will be readily understood, a more particular description of the invention briefly described above will be rendered by reference to specific embodiments illustrated in the appended drawings. Understanding that these drawings depict only typical embodiments of the invention and are not therefore to be considered limiting of its scope, the invention will be described and explained with additional specificity and detail through use of the accompanying drawings, in which:

FIG. 1 is a flow diagram of systems and methods in accordance with an embodiment of the invention;

FIG. 2 is a flow diagram of systems and methods in accordance with an embodiment of the invention;

FIG. 3 is a screen view of a device and method in accordance with an embodiment of the invention;

FIG. 4 is a flow diagram of systems and methods in accordance with an embodiment of the invention;

FIG. 5 is a screen view of a device and method in accordance with an embodiment of the invention;

FIG. 6 is a functional diagram of systems, devices, and methods in accordance with an embodiment of the invention;

FIG. 7 is a functional diagram of systems, devices, and methods in accordance with an embodiment of the invention;

FIG. 8 is a functional diagram of systems, devices, and methods in accordance with an embodiment of the invention;

FIG. 9 is a functional diagram of systems, devices, and methods in accordance with an embodiment of the invention;

FIG. 10 is a functional diagram of systems, devices, and methods in accordance with an embodiment of the invention;

FIG. 11 is a flow diagram of systems and methods in accordance with an embodiment of the invention;

FIG. 12 is a flow diagram of systems and methods in accordance with an embodiment of the invention;

FIG. 13 is a flow diagram of systems and methods in accordance with an embodiment of the invention;

FIG. 14 is a flow diagram of systems and methods in accordance with an embodiment of the invention;

FIG. 15 is a flow diagram of systems and methods in accordance with an embodiment of the invention; and

FIG. 16 is a flow diagram of systems and methods in accordance with an embodiment of the invention.

### DETAILED DESCRIPTION

It will be readily understood that the components of the present invention, as generally described and illustrated in the Figures herein, may be arranged and designed in a wide variety of different configurations. Thus, the following more detailed description of the embodiments of the invention, as represented in the Figures, is not intended to limit the scope of the invention, as claimed, but is merely representative of certain examples of presently contemplated embodiments in accordance with the invention. The presently described embodiments will be best understood by reference to the drawings.

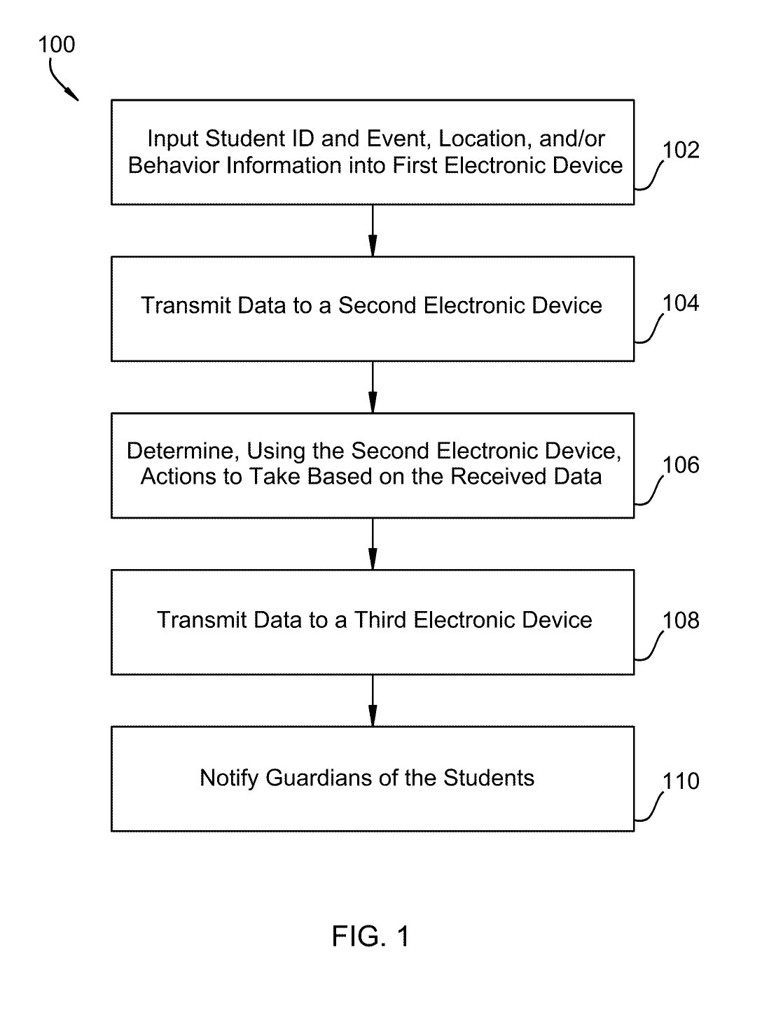

FIG. 1 show a flow diagram 100 including steps 102-110. At step 102, student identification information and event information, location information, and/or behavior information is input or received at a first electronic device. The first electronic device may be a student device, guardian device, school device, of staff device. First devices may be mobile or fixed devices, include GPS location technology, and be owned by students, guardians, schools, or governments. First electronic devices may include computer systems, iPads, iPods, smartphones, computer kiosks, laptops, notebooks, or PDAs. Student identification information and devices (Student ID) may be school identification cards, school identification numbers, student names, student assigned barcodes, dynamically linked barcodes, barcodes with embedded student identifiers, student phone numbers, student email addresses, electronic identification cards, student devices, hardware identifiers on student devices, software identifiers on student devices, network identifiers on student devices, or a combination thereof. A student identifier may be linked to a biometric identifier such as fingerprints of the student, facial features, voice features, and combinations thereof. Event data may be, in some embodiments, location data where the student ID was input, scanned, read, or manually input into the first electronic device. In other embodiments, event data may be data about a time stamped event such as entering school property, leaving school property, entering a class room, eating lunch, getting on the bus, getting off the bus, entering a doorway, leaving a doorway, walking down a hall, or sitting in a chair. In other embodiments, event data may be linked to events such as walking, running, sitting, standing, yelling, crying, not moving for a predetermined amount of time, rate of respirations, heart rate, blood pressure, or a combination thereof. In other embodiments, combinations of the above event data embodiments may be used together to provide event data combinations. Location information may include a location of a scan of a student ID, location of a manual input of a student identifier, network location identifier, school location identifier, a fixed location of a student ID input device, an owner or user of the input device (such as a specific mobile guardian device or school staff device), or a combination thereof. Behavior information may include visual behavior, school work behavior, bullying behavior, emotional behavior, physical behavior, group behavior, or combinations thereof.

At step 104, the student identification information along with one or more of event information, location information, time information, and/or behavior information is transmitted to a second electronic device. The second electronic device may be a computer, server, database, database server, cloud computing system, enterprise data system, local network computer system, wide area computer system, or a combination thereof. The data may be transmitted using wireless, Wi-Fi, Internet, optical fiber, ethernet, Bluetooth, RFID, NFC, or a combination thereof. At step 106, programming in the second electronic device is used to determine one or more actions to take based on the received data. The one or more actions may include storing data, sending one or more notifications, updating one or more privileges of students, allowing school staff to access information about one or more students, allowing guardians to access information, or a combination thereof. Data may be store on device or computer when no Internet is available and transmitted to cloud or server when device enters a Wi-Fi or Internet area.

At step 108, information is transmitted to a third electronic device. The third electronic device may be a student device, guardian device, school device, of staff device which a guardian is logged into. The third electronic device may include computer systems, iPads, iPods, smartphones, computer kiosks, laptops, notebooks, or PDAs. The transmitted data 108 may include interactive instructions, selections, and/or notifications for guardians of students from the student, other guardians, guardians of other students, and/or school support staff. The notifications 110 may include confirmations of interactive actions taken by guardians based on selections and prompts from the second electronic device.

In one example, a student (Robert) may report to his guardian (Roberta) that another student (Frank) threatened him today during lunch. Roberta may then log in to a school communication system using a program application on her phone. Once logged in, Student ID information (Guardian of Robert) and Location data 102 (authenticated, located, and identified using her mobile phone application) is transmitted along with bullying behavior information of an encounter of Frank and Robert to second electronic device 104. The encounter information may include other information such as guardian concern level, time, date, guardian suggestions, location information of encounter, shared classes between Robert and Frank, a request for a school meeting, or a combination thereof. Then the second electronic device determines 106, based on programming, how to proceed with actions, notifications, and automated responses. In this example, the system determines to alert the science teacher 108, Mrs. Ortega, that an increased awareness is requested in observing interactions between Robert and Frank, send a notification 108 to the school vice principle, and to guardians of both Robert and Frank based on the reporting of Roberta 108. Additionally, a confirmation notification may be sent Roberta alerting her that action has been taken and what type of action 110.

In a second example, a barcode on a student ID is scanned at a football game 102. Location information (football stadium, Forest High School), time information, and a student identifier are sent to second electronic device 104. A determined action 106 may be that the student is barred from attending football games because of behavior, conduct, grades, or reasons. Another determined action may be to notify a guardian of the attempt to attend a football game with the time and location. Notifications may be sent by text message, SMS, push notifications, email or by other instant messaging/social media systems such as Facebook, Twitter, Snapchat, or a combination thereof. A notification may include reasons why the student was barred and steps necessary to correct the barred state. In a third example, a student enters school property and a push notification from a student smart phone asks the student to authenticate their identity. The student may use recognition features found on a student smartphone, iPad, iPod, laptop, or computer, school kiosk, school computer to authenticate their identity using a finger print identification device, camera facial recognition, login with a user name and password, use a camera to scan a barcode on an ID card of the student, or use an NFC chip embedded in an ID card of the student. The ID card may be a school ID card or a student credit card with NFC capabilities. A student NFC enabled ID may be placed near a student smartphone or other computer device to authenticate a student's identity. The identity information along with location, event or behavior information may be transmitted to a second electronic device 104.

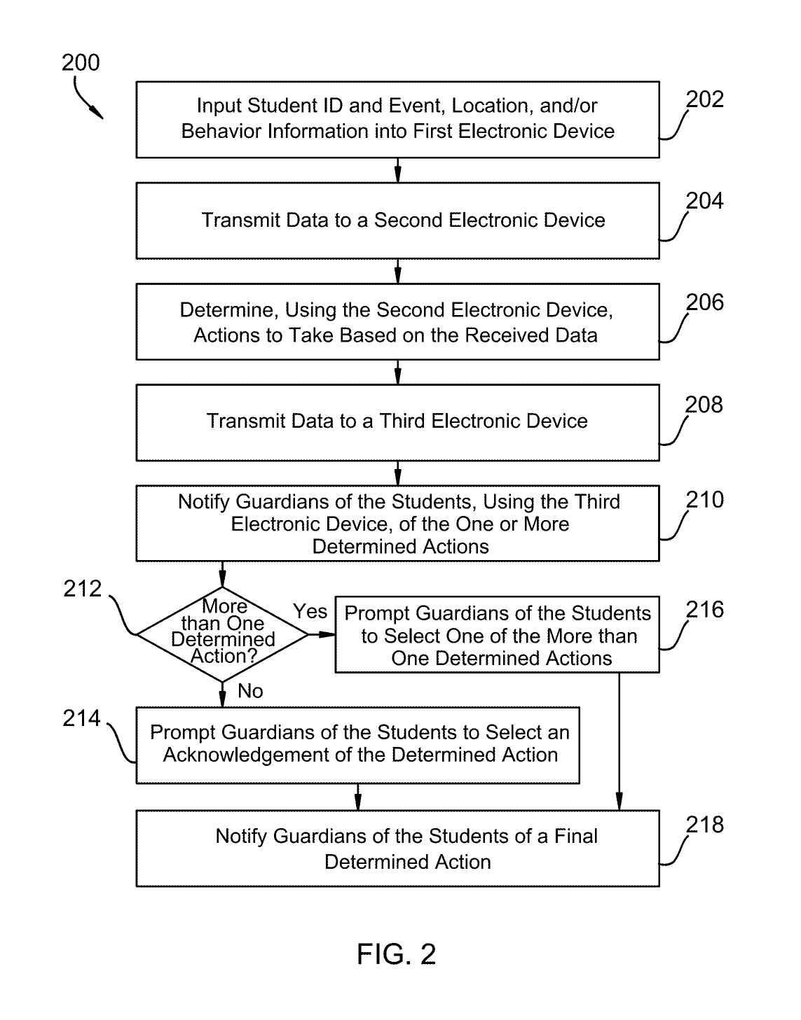

FIG. 2 show a flow diagram 200 including steps 202-218. At step 202, student identification information and event information, location information, and/or behavior information is input or received at a first electronic device. The first electronic device may be a student device, guardian device, school device, of staff device. First devices may be mobile or fixed devices, include GPS location technology, and be owned by students, guardians, schools, or governments. Student identification information and devices (Student ID) may be school identification cards, school identification numbers, student names, student assigned barcodes, dynamically linked barcodes, barcodes with embedded student identifiers, student phone numbers, student email addresses, electronic identification cards, student devices, hardware identifiers on student devices, software identifiers on student devices, network identifiers on student devices, or a combination thereof. A student identifier may be linked to a biometric identifier such as fingerprints of the student, facial features, voice features, and combinations thereof. Event data may be, in some embodiments, location data where the student ID was input, scanned, read, or manually input into the first electronic device. In other embodiments, event data may be data about a time stamped event such as entering school property, leaving school property, entering a class room, eating lunch, getting on the bus, getting off the bus, entering a doorway, leaving a doorway, walking down a hall, or sitting in a chair. In other embodiments, Event data may be linked to events such as walking, running, sitting, standing, yelling, crying, not moving for a predetermined amount of time, rate of respirations, heart rate, blood pressure, or a combination thereof. In other embodiments, combinations of the above event data embodiments may be used together to provide event data combinations. Location information may include a location of a scan of a student ID, location of a manual input of a student identifier, network location identifier, school location identifier, a fixed location of a student ID input device, an owner or user of the input device (such as a specific mobile guardian device or school staff device), or a combination thereof. Behavior information may include visual behavior, school work behavior, bullying behavior, emotional behavior, physical behavior, group behavior, or combinations thereof.

At step 204, the student identification information along with one or more of event information, location information, time information, and/or behavior information is transmitted to a second electronic device. The second electronic device may be a computer, server, database, database server, cloud computing system, enterprise data system, local network computer system, wide area computer system, or a combination thereof. The data may be transmitted using wireless, Wi-Fi, optical fiber, ethernet, Bluetooth, RFID, NFC, or a combination thereof. At step 206, programming in the second electronic device is used to determine one or more actions to take based on the received data. The one or more actions may include storing data, sending one or more notifications, updating one or more privileges of students, allowing school staff to access information about one or more students, allowing guardians to access information, or a combination thereof.

At step 208, information is transmitted to a third electronic device. The third electronic device may be a student device, guardian device, school device, of staff device which a guardian is logged into. The third electronic device may include computer systems, iPads, iPods, smartphones, computer kiosks, laptops, notebooks, or PDAs. The transmitted data 208 may include interactive instructions, selections, and/or notifications for guardians of students from the student, other guardians, guardians of other students, and/or school support staff. The notifications 210 may include confirmations of interactive actions taken by guardians based on selections and prompts from the second electronic device. At step 212, a determination is made if more than one action will be taken allowing the interactive participation of the guardian of the student. If a guardian needs to make an interactive selection, then a push notification may be sent to a smartphone or other guardian device of the student asking for a selection 216. After the guardian makes a selection, a confirmation notice of a final action may be sent to the guardian's device 218. If no selection of an action is needed by the guardian, a an acknowledgement of the action may be sent to a guardian device 214 and after acknowledged by the guardian, a confirmation of receipt of the notice and a final determined action may be sent out to the guardian device 218.

In one example, a guardian may receive a push notice to their device that they are required to attend a mandatory meeting with their student and have options for dates and times for the meeting. After selection of a date and time, a final notice of the meeting time and date may be sent as a final confirmation to the guardian device.

In another example, a guardian may receive a push notification that their daughter has earned 2 hours of detention and optional times and dates are pushed for the parent to select. After selection is made, a final confirmation notice of the dates and times may be sent to the guardian device.

In another example, all guardians of students may be invited to a school assembly. A push notification may be sent out and received by the guardian device and acknowledged by the guardian. If the push notification is not acknowledged, an automated phone call or email may follow. After acknowledgement of the push notification, a final notification may be sent to the guardian device with a confirmation of attendance or a confirmation of declining attendance or a “thanks for acknowledging” message.

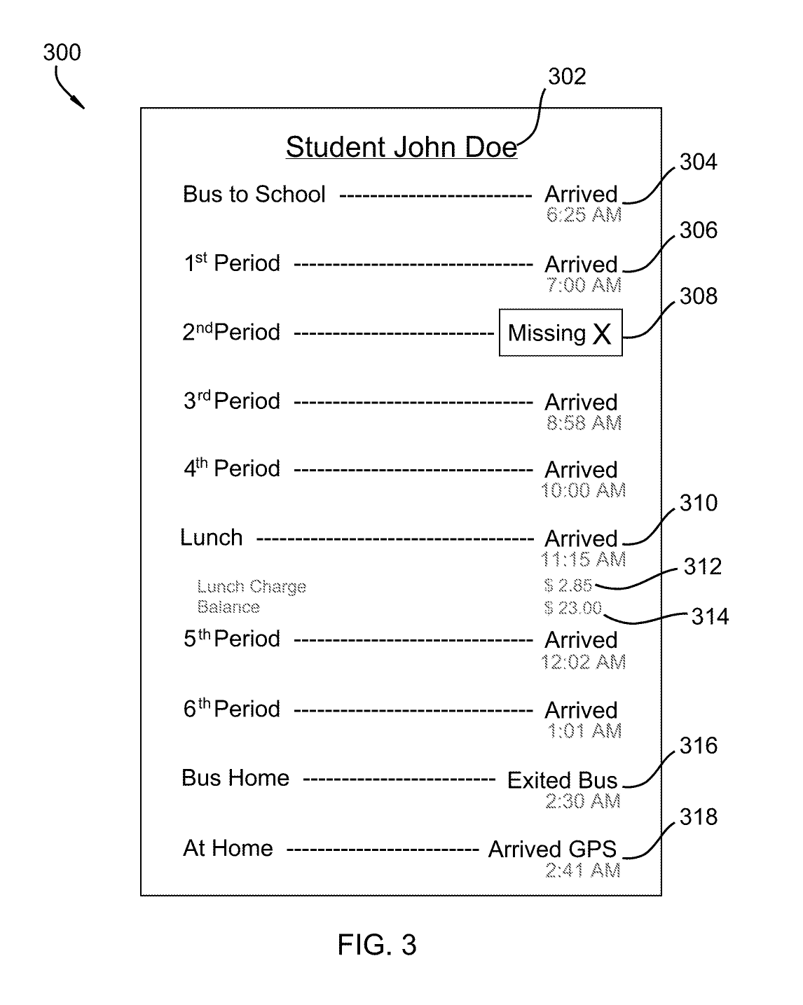

FIG. 3 depicts a screen shot 300 of a daily log of a student 302 on a student device, guardian device, school device, and/or staff device. Screen shot 300 includes a student name 302, a time when student 302 arrived on the school bus 304, a time the student arrived at firs period 306, a missed period 308, a time arrived at lunch 310, an amount spent at lunch 312, a positive lunch balance 314, a time exiting the bus 316 and a time arriving at home 318. Events listed here are examples of reporting based on time and location of student events. These and other events may be made easily available to guardians, school staff, law enforcement, and the students through application software running on student device, guardian devices, school devices, and staff devices. Indicators such as missing 308 may be texted or sent by push notifications to guardians and school staff. Automated reporting and student privilege granting may be associated with student events and timing of events. Events may be logged and monitored by GPS and reported through application software or by data inputs as described in relation to FIG. 1.

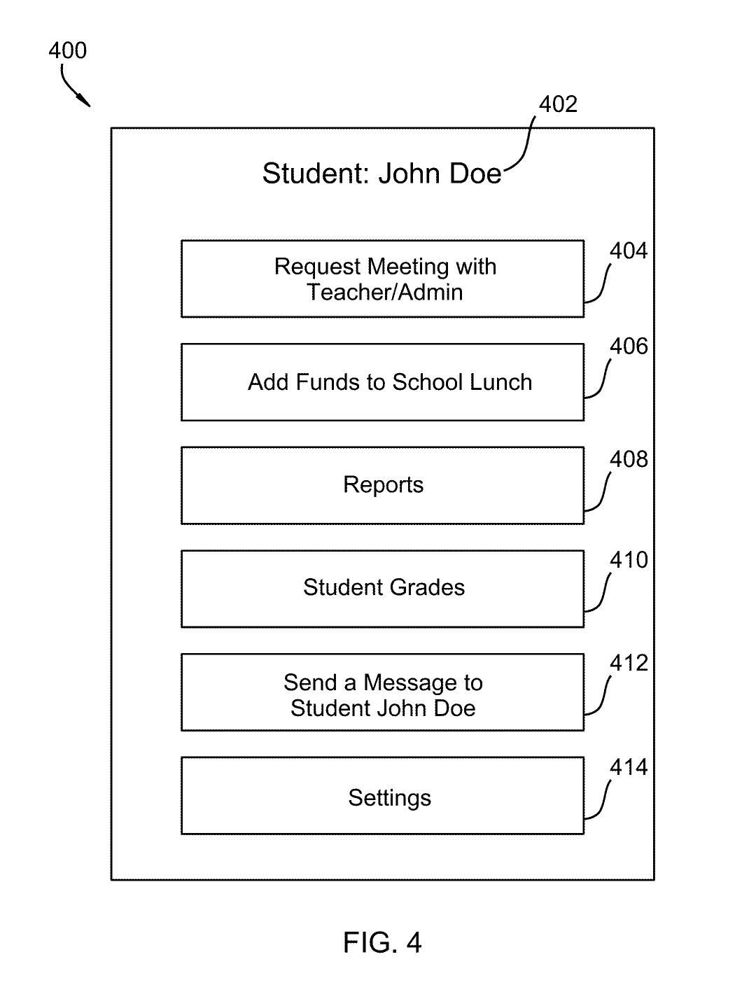

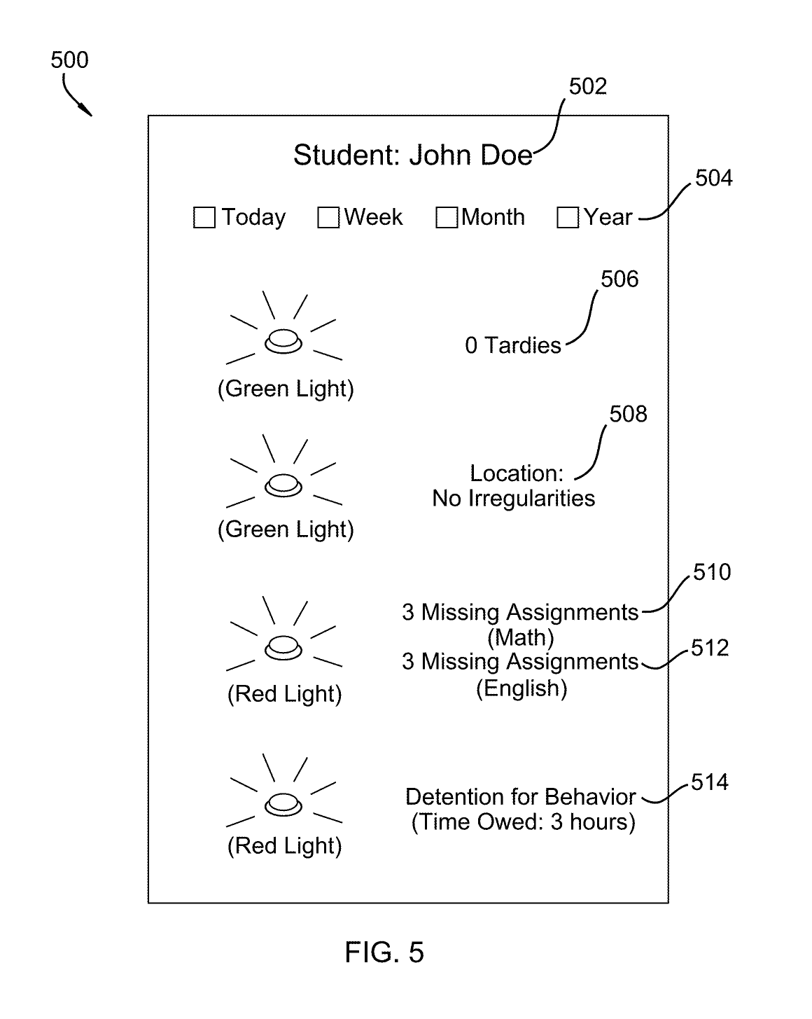

FIG. 4 shows an example dashboard screenshot 400 of a student, guardian, school or staff device. Name 402 may be a student name, guardian name, staff, name, or school name. Menus 404-414 may be customized through settings menu 414 or through backend programming menus (not shown). In the specific example of FIG. 4, an example guardian menu 400 is shown for student John Doe 402. A guardian may request a meeting with a teacher, staff, administration 404. Funds may be added to a school lunch account 406. Various reports such as the example report shown FIGS. 3 and 5 may be accessed through report menu 408. Student grades 410 including missing assignments may be accessed. An option for a guardian to send a message to their student 412 through text, push notifications, email, voice message, direct call, or a hand delivered note is available through menu 412. Various settings for displaying menus, notification configuration, reports, content, and automatic features are included in settings menu 414. Settings menus may be different for different users. Student, guardian, and staff menus may contain functionality commensurate in a scope of job function, legal regulations, school policies, grade level, and specific needs-based settings.

FIG. 5 shows an example dashboard screenshot 500 of a student, guardian, school or staff device. Name 502 may be a student name, guardian name, staff, name, or school name. Indicators 506-514 may be customized through settings menu 414 of FIG. 4 or through backend programming menus (not shown). In the specific example of FIG. 5, an example of a guardian quick view dashboard indicators 500 is shown for student John Doe 502. Dashboard indicators may be configured for guardians, student, or staff. For student John Doe 502, a time period selection 504 is selected to view key behavior indicators 506-514 within the selected time period. Key behavior indicator 506-514 may be customized according to school, guardian, staff, or students needs, desires, and requirements. Here in this specific example, John has 0 tardies 506, no location irregularities 508, missing assignments 510,512 and 3 hours of owed detention 514. Each behavior indicator may be selected, and a report generated with specific dates, times, locations, assignments, classes, etc. for the selected time period.

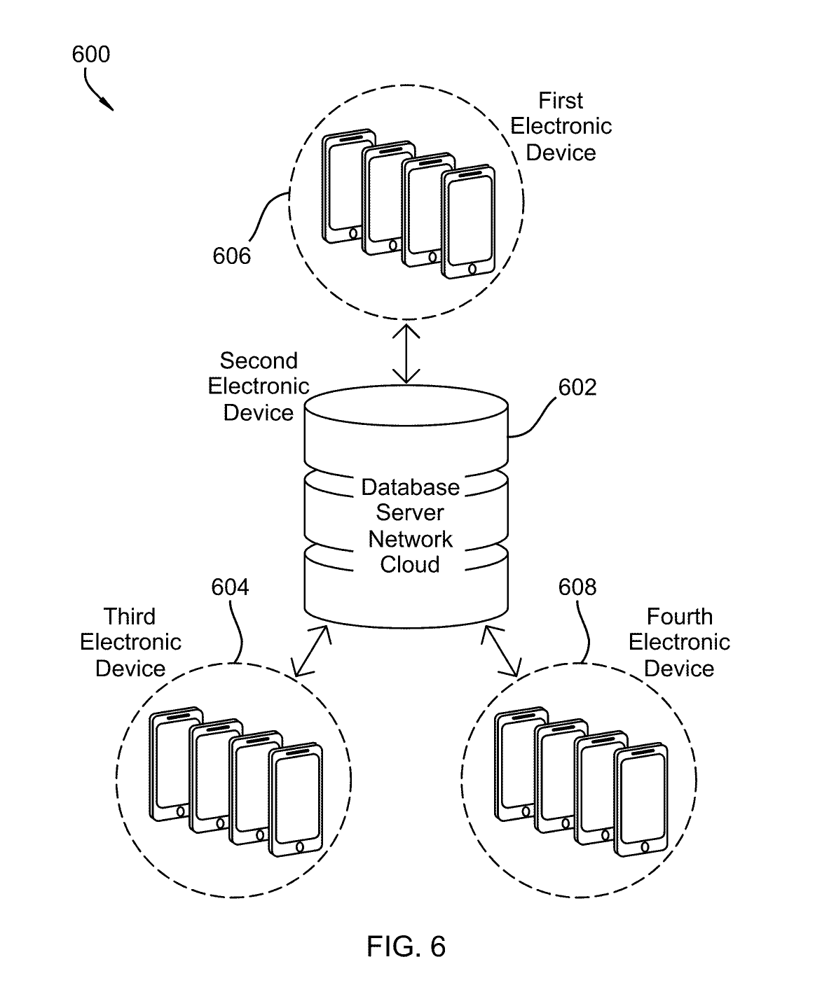

FIG. 6 shows a functional reporting diagram 600 of communications between students, guardians, and staff. First electronic devices 606, second electronic devices 602, third electronic devices 604, and fourth electronic devices 608 may be connected by a combination of wired and wireless networks. The networks may be local, wide-area, or cloud-based networks. Communication may take place over the Internet or be a private closed network, or a combination thereof. Some reporting and communication features may be accomplished while not connected to a network and perform syncing operations when a network is available. This syncing feature may take place on any of the First, Third, or Fourth communication devices.

First communication devices 606 may include a student device, guardian device, school device, of staff device. First devices may be mobile or fixed devices, include GPS location technology, and be owned by students, guardians, schools, or governments. First electronic devices may include computer systems, iPads, iPods, smartphones, tablets, computer kiosks, laptops, notebooks, or PDAs.

Third communication devices 604 may include a student device, guardian device, school device, of staff device. Third devices may be mobile or fixed devices, include GPS location technology, and be owned by students, guardians, schools, or governments. Third electronic devices may include computer systems, iPads, iPods, smartphones, tablets, computer kiosks, laptops, notebooks, or PDAs.

Fourth communication devices 608 may include a student device, guardian device, school device, of staff device. Fourth devices may be mobile or fixed devices, include GPS location technology, and be owned by students, guardians, schools, or governments. Fourth electronic devices may include computer systems, iPads, iPods, smartphones, tablet, computer kiosks, laptops, notebooks, or PDAs.

Second electronic communication devices 602 may be a computer, server, database, database server, cloud computing system, enterprise data system, local network computer system, wide area computer system, or a combination thereof. Data to and from the second communication device 602 may be transmitted using wireless, Internet, Wi-Fi, optical fiber, ethernet, Bluetooth, RFID, NFC, or a combination thereof.

Groups of first devices, third devices, and fourth devices may each have unique properties, privileges, functionality, and settings unique to the group. Additional divisions in privileges, functionality, and settings may be sub-divided within the parameters of each group for specific individual users of the group.

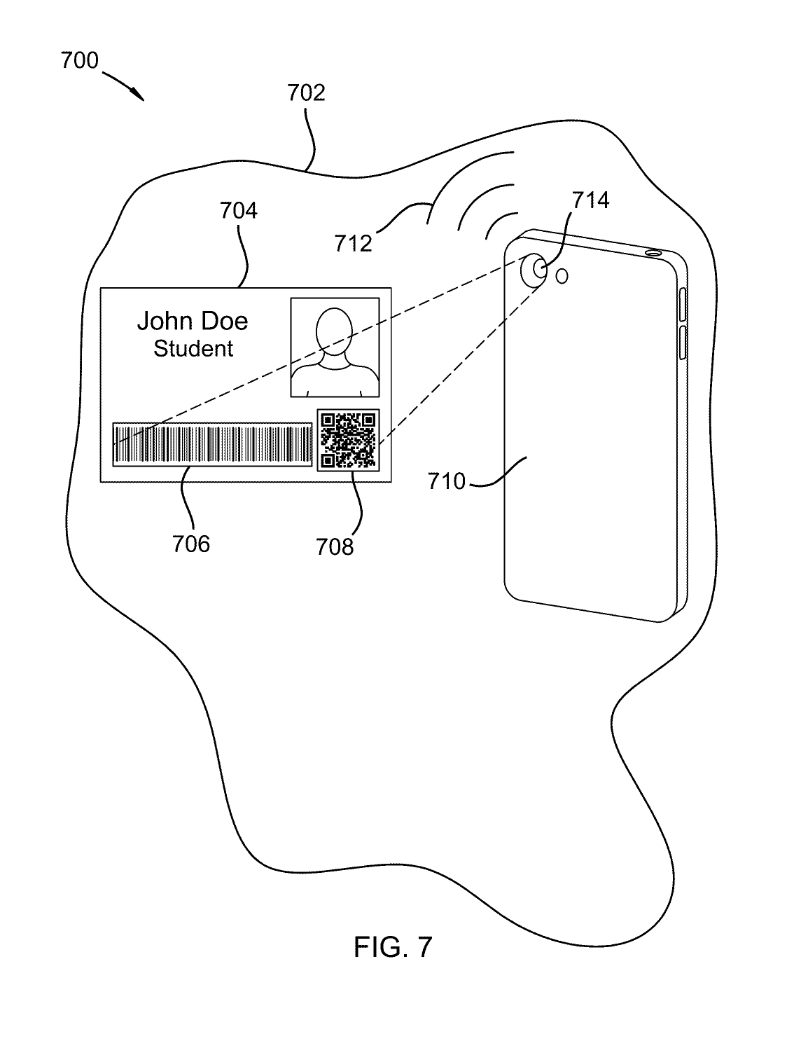

FIG. 7 shows a method 700 of data input using a smartphone 710 to capture a barcode on student ID 704. Device 710 is represented graphically as a smartphone but may be computer systems, iPads, iPods, tablets, smartphones, computer kiosks, laptops, notebooks, or PDAs. Student ID 704 may be a digital student ID on a students' phone or a physical student ID card. Smart phone 710 may be a staff device, guardian device, or a student device.

In one example, a student arrives on school campus 702 and desires to login to a school wireless network 712. The student opens an application program on his smartphone 710 and scans a barcode 706/708 on his student ID. Barcodes 706/708 may contain specific login URLs, credential, user names, password, access keys to allow the student access to a school wireless network 712 after the student scans one or more barcodes 706/708. The school wireless network may give the student network access only through the student application software on the students' device 710. The school may record attendance, notify guardians of the student of the arrival at school of the student as a result of the student scanning the barcode. The school may record attendance, notify guardians of the student of the arrival at school of the student as a result of the student logging into the school's network. GPS location services on the students' device may track the students location throughout a school day using specific school application software installed on the students' phone.

In another example, a staff device 710 scans a student ID card 704 in a hallway at school using the schools network 712 to record the student tardy. A notification may be generated and sent to one or more guardian or staff devices to record or notify an individual of the tardy. The tardy may also be recorded in a school behavior record and possibly impact the ability of the student to use physical/software/digital school privileges.

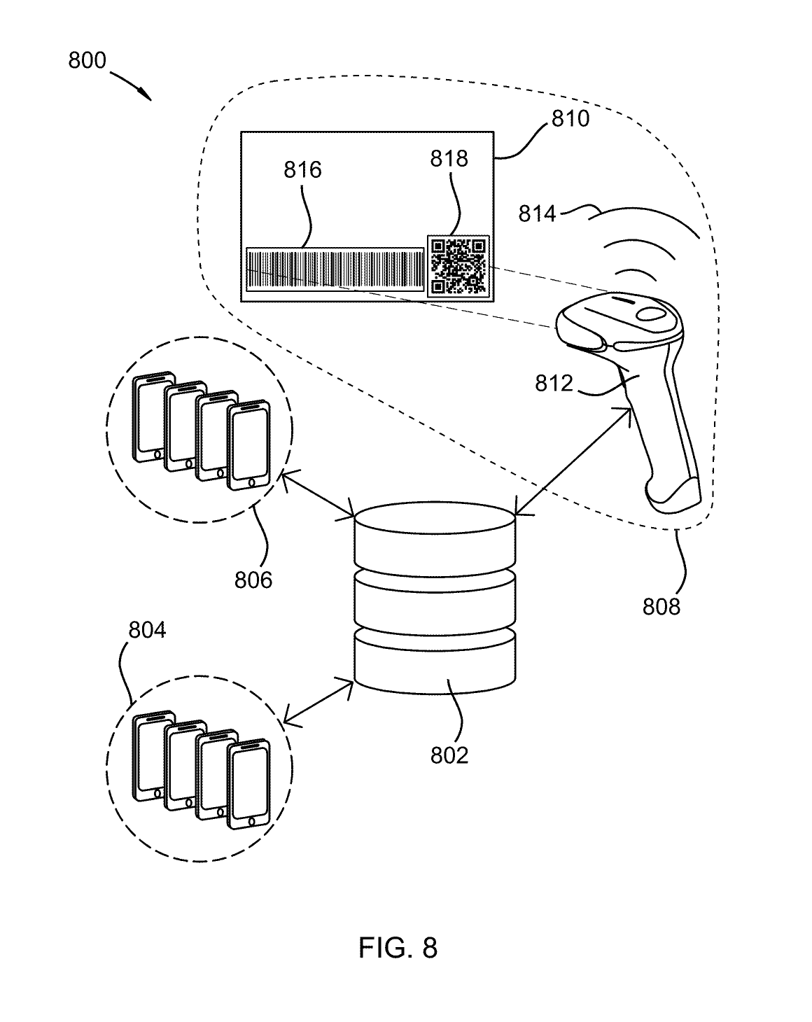

FIG. 8 shows a communication system 800 including one or more wireless or wired barcode scanners 812 providing data input to second communication system 802. Device 812 is represented graphically as a wireless (scanner school device) but may be a computer system, iPad, iPod, smartphone, computer kiosk (school device), laptop, notebook, or PDA device. Communication devices 806 and 804 may be staff devices, student devices, or guardian devices. Scanner 812 may be located within a fixed area wireless network 814 or fix region 808 such as a school campus. ID 810 may be a digital ID of staff, students, or guardians and contain one or more barcodes 816/818. Barcodes 816/818 may be used to authenticate staff, students, and/or guardians.

In one example, a school staff member desires to communicate with a guardian through a school communication system. The staff member scans his ID 810 and is authenticated into the school communication system and has access to send a push message to a guardian device.

In another example, a staff member is scanning student IDs with scanner 812 as the student arrives or departs at school dance. The guardians of the students may be notified of the arrival/departure of their student to and/or from the dance.

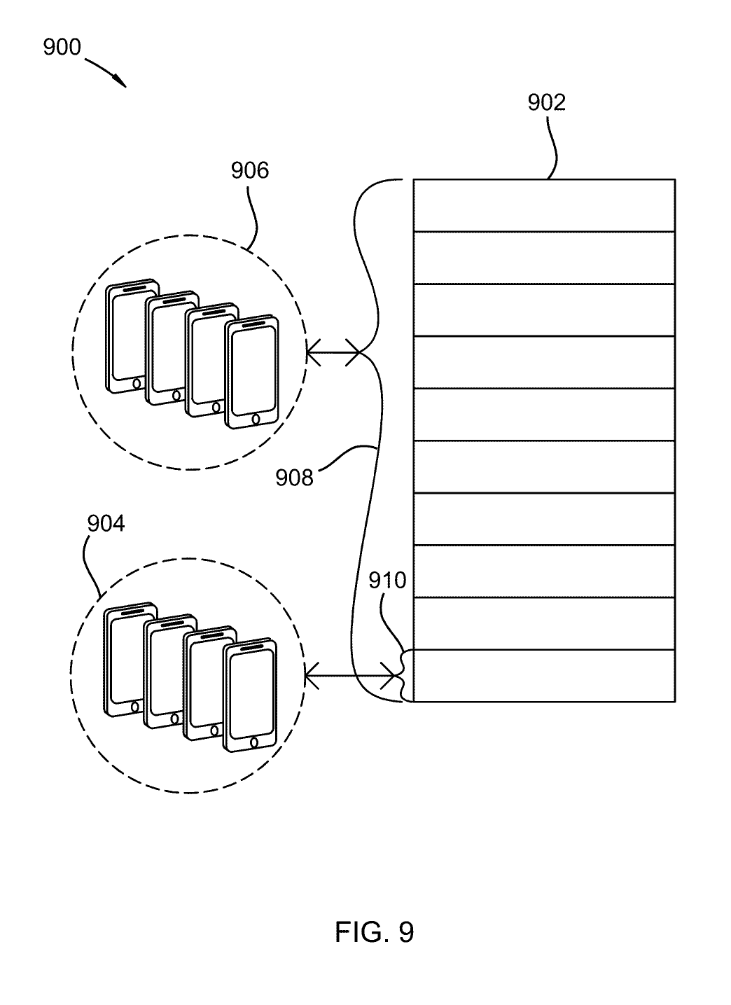

FIG. 9 shows a representation of student privileges 900 between groups of students 906 and 904. Student group 906 may have specific privileges 908 which are different from specific privileges of student group 904. Student school privileges 902 may consist of network privileges, software privileges, digital use privileges, and physical use privileges. Privileges may be assigned to student groups based on behavior of students. Students with missing assignments 904 may, for example, be restricted from using streaming music network functionality while at school. Other restrictions may include revoking passes to leave for lunch, revoking access to digital games, reducing or revoking network speed while at school, school sporting event attendance, school social event attendance, school clubs, school chat systems, school social media, etc. Privileges may be divided into two or more groups. Student privileges may be used as a motivational indicator on student devices to help student get homework turned in or to assist in positive behavior modification. Notifications of student privileges may be sent to guardian device, staff devices, and student devices.

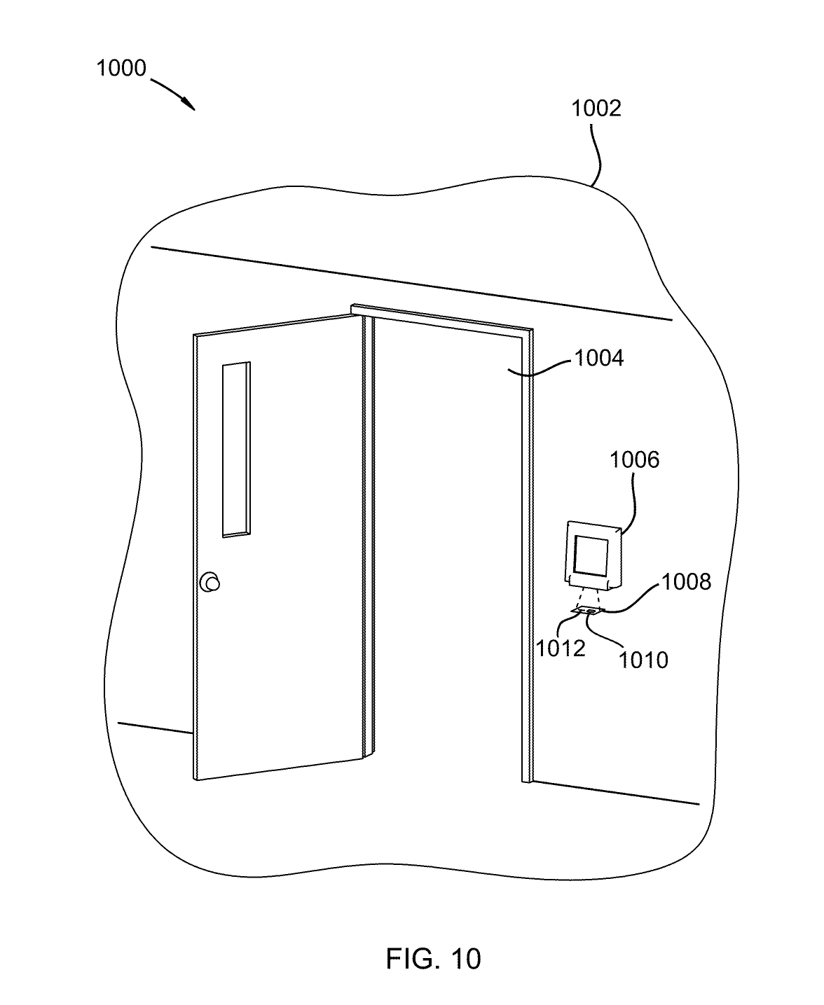

FIG. 10 shows an example of a school kiosk device 1006. Kiosk 1006 may be attached to a school structure 1002. School structure 1002 may be a school bus, class room, doorway, hallway, cafeteria, walls, ceilings, or any other physical structure. Kiosk 1006 may include optical scanners for scanning barcodes, NFC interrogators for reading NFC ID cards, Wireless access points for communication with student, staff, and guardian devices, RFID interrogators for interrogating RFID enabled ID cards within a particular area.

In one example, a student scans one or more barcodes 1012/1010 as the student enters a doorway 1004 of classroom 1002 and places his ID card proximate to an optical scanner of kiosk 1006. In other embodiments kiosk 1006 may wirelessly communicated with a student device as the student walks past the kiosk using Bluetooth or another wireless communication technology. In another embodiment, kiosk 1006 unobtrusively and unknown to the student, wirelessly interrogates a student card as the student walks through the doorway using RFID technology.

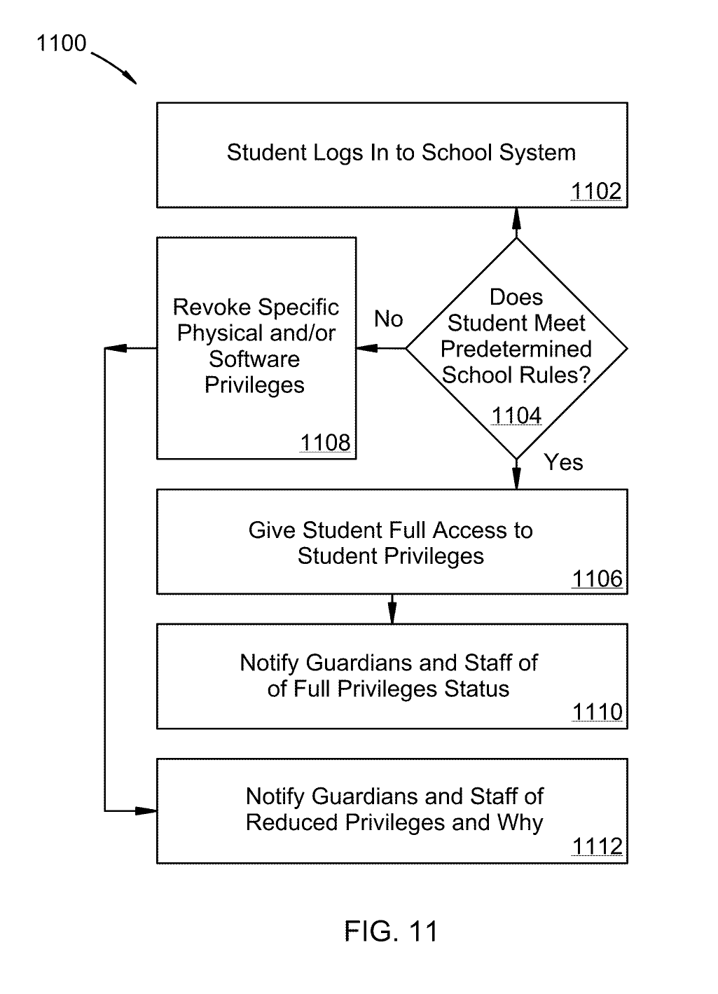

FIG. 11, shows a flow diagram 1100 of systems and methods in accordance with an embodiment of the invention. At step 1102 student identification information is received and a student is logged into a school network. This log in may be the result of the student being in range of the school network on the students' device, scanning a barcode of a student ID by the student or by a staff member, or a manual login by either a student or staff member. Student identification information and devices (Student ID) may be school identification cards, school identification numbers, student names, student assigned barcodes, dynamically linked barcodes, barcodes with embedded student identifiers, student phone numbers, student email addresses, electronic identification cards, student devices, hardware identifiers on student devices, software identifiers on student devices, network identifiers on student devices, or a combination thereof. A student identifier may be linked to a biometric identifier such as fingerprints of the student, facial features, voice features, and combinations thereof. At step 1104, a determination is made if the student is compliant with predetermined school rules. If student is compliant, access privileges are given 1106. Access privileges may be customized for groups of students in similar grades, ages, schools, etc. Access privileges may include physical privileges such as school activities, rallies, sporting events, field trips. Access privileges may also include digital privileges such as network access, network speed, software access, social media access, music access, etc. At step 1108, when a student is non-compliant with predetermined school rules, access privileges may be reduced or revoked. Behavior information may include visual behavior, school work behavior, bullying behavior, emotional behavior, physical behavior, group behavior, or combinations thereof. Guardian of students may receive notifications of a students' access privileges and updates as the access privileges change 1110. If privileges are revoked or reduced, a notification may be sent to the guardian informing the guardian of the changes and what the student needs to do the have the privileges restored 1112.

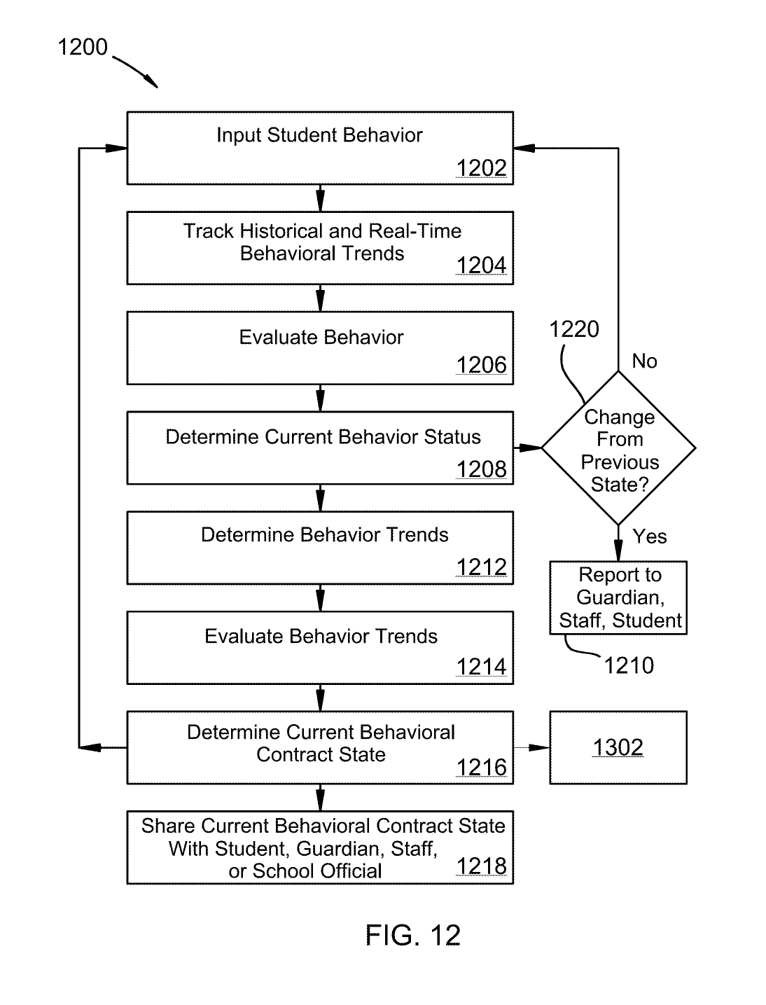

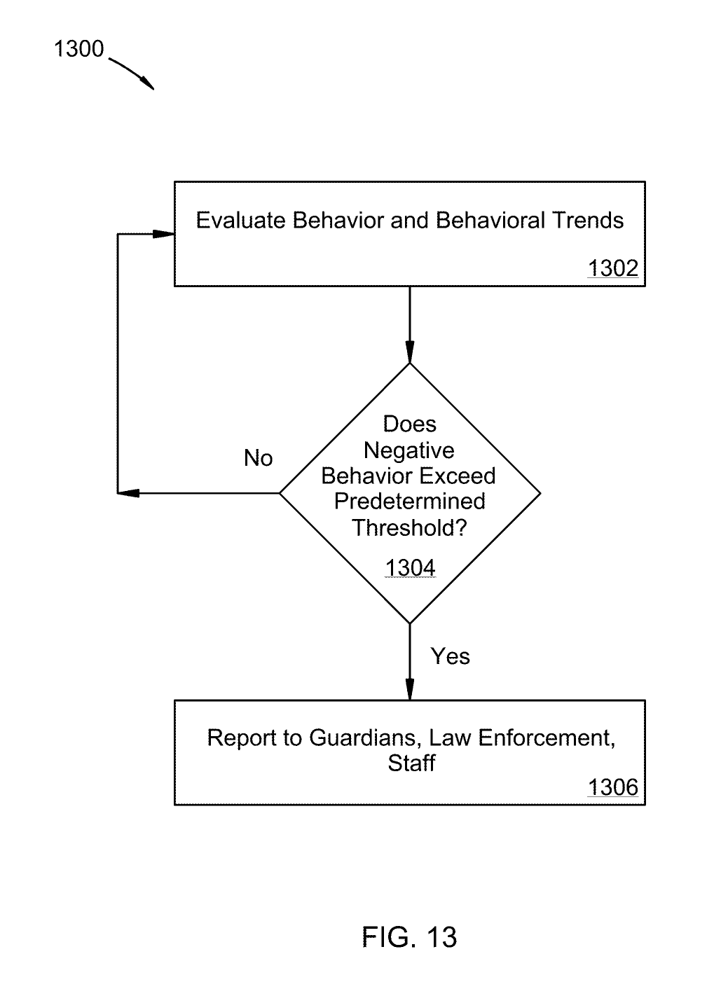

FIG. 12 shows a flow diagram 1200 of systems and methods in accordance with an embodiment of the invention. At step 1202 student behavior is input into a school communication system. Behavior information may include visual behavior, school work behavior, bullying behavior, emotional behavior, physical behavior, group behavior, or combinations thereof. Input of behavior may come from a student device, guardian device, school device, of staff device. At step 1204, historical and real-time behavior trends are tracked. The tracking may include time stamping events, locations, and the method of receiving behavior data. Behavioral event data may be linked to events such as walking, running, sitting, standing, yelling, crying, not moving for a predetermined amount of time, rate of respirations, heart rate, blood pressure, or a combination thereof. In other embodiments, combinations of the above event data embodiments may be used together to provide event data combinations. Location information may include a location of a scan of a student ID, location of a manual input of a student identifier, network location identifier, school location identifier, a fixed location of a student ID input device, an owner or user of the input device (such as a specific mobile guardian device or school staff device), or a combination thereof. At step 1206, the behavior data is evaluated. This evaluation may be an automatic programmed evaluation to find multiple behavior event within a predetermined time period, multiple reports from a single source, multiple reports from multiple sources, law enforcement reports, sources of reports (other students, the student himself, guardians, staff), and combinations thereof. At step 1208, a current behavior status is determined. This may be a result of recent behavior entries. Recent behavior entries may indicate depression, suicide risk, violence risk, risk to others, etc. A current behavior status may trigger 1220 notifications to be sent to student devices, staff devices, and guardian devices 1210. At step 1212 trends of behavior are determined and compared to a behavior contract state 1216. Behavior contract states may be contracts between specific students and schools/staff and/or general contacts between a school and all students in a school. Such contracts may be found in documents like school handbooks. Behavior contract states are further described in relation to FIG. 13 (1302). At step 1218, a notification is sent to a student device, guardian device, or staff device detailing a behavioral contract state of a student. A notification may include such information as “your student is fully compliant with all aspect of school policy” or may include specific areas that the student is non-compliant with school policy or a specific individual student contract plan.

FIG. 13 shows a flow diagram 1300 of systems and methods in accordance with an embodiment of the invention. At step 1302, an evaluation of a students' behavioral trend is compared to predetermined thresholds 1304 to determine notifications to send out 1306.

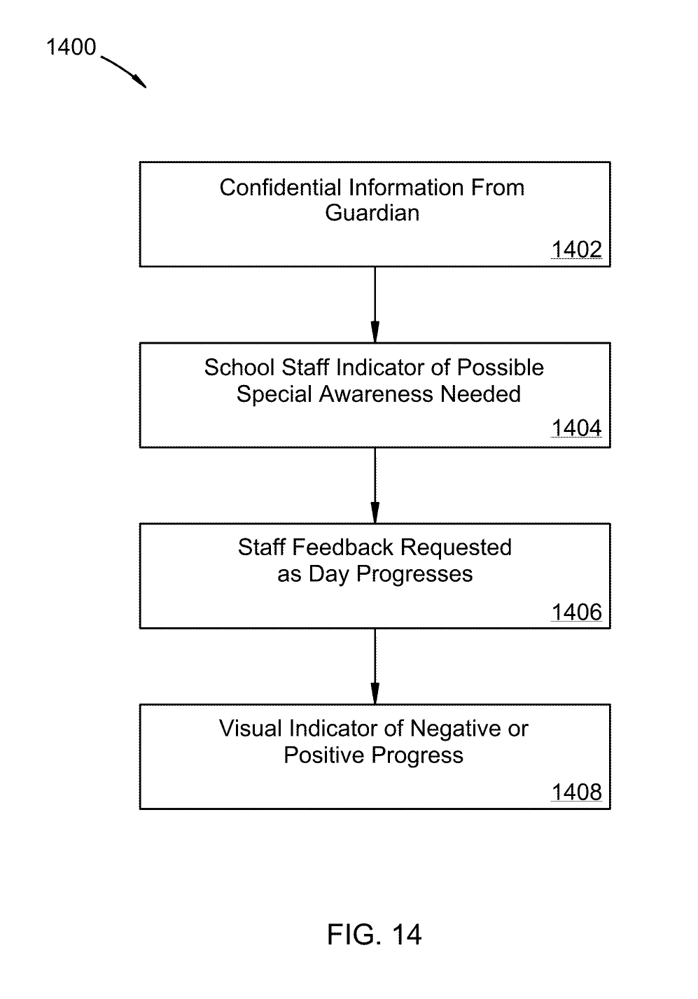

FIG. 14 shows a flow diagram 1400 of systems and methods in accordance with an embodiment of the invention. At step 1402, confidential information is conveyed from a guardian to a school communication system. At step 1404, an automatic notification may be sent to student devices, guardian devices, and staff devices of a need to pay attention or give additional awareness to a particular student or situation. At step 1406, staff may be requested to comment or annotate behavior of a specific student or situation. At step 1408, guardians, staff, and/or students may receive or be able to see negative or positive progress related to a particular situation or student(s).

In one example, a guardian reports confidential information to a communication school system that their student is threatening suicide 1402. A school notification may be automatically sent out to staff, students and/or guardians that a special awareness is needed in regard to the student and ask for specific feedback on student daily interactions 1406 without disclosing the confidential information. Visual indicators of behavioral progress may be made available or pushed to guardian, staff, and/or student devices 1408.

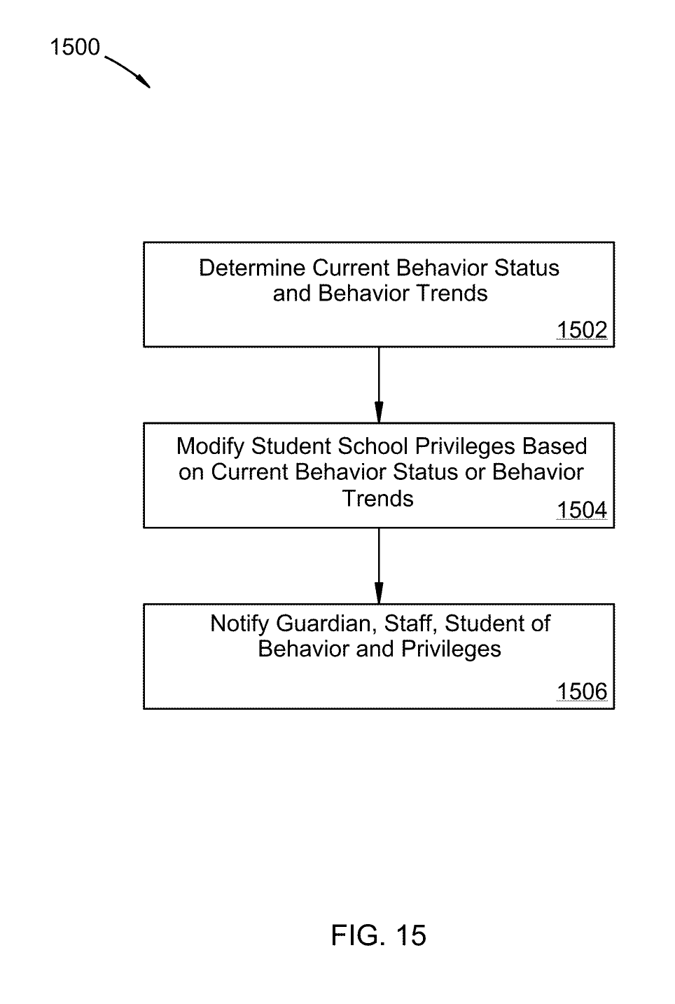

FIG. 15 shows a flow diagram 1500 of systems and methods in accordance with an embodiment of the invention. At step 1502, a current behavior status and/or behavior trends may automatically modify student school privileges 1504. Guardians, staff, and students may be notified of changes in school privileges.

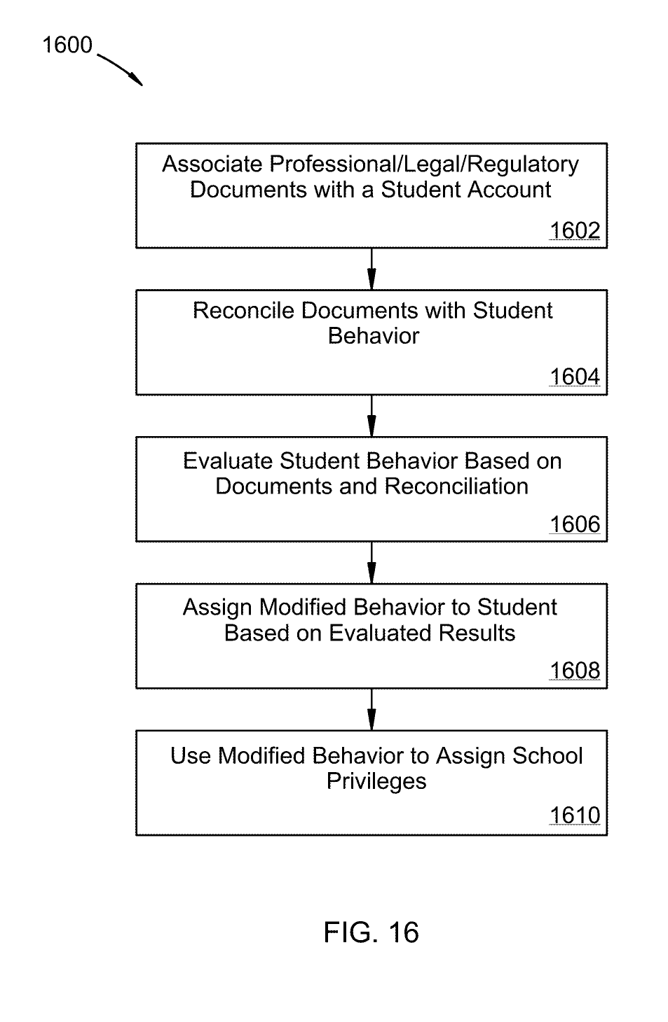

FIG. 16 shows a flow diagram 1600 of systems and methods in accordance with an embodiment of the invention. At step 1602, professional, legal, regulatory, school contracts, etc., are associated with a student school account. The attached documents may be used to evaluated behavior of the student 1604-1606. A determined behavior may be assigned or modified based on the attached documents 1608. The assigned or modified behavior may be used to assign school privileges 1610.

In one example, a student is mentally challenged and has professional documentation detailing a psychiatric medical condition. When this student was found intentionally dumping water on a floor in a school way, a teacher scanned his ID and was immediately informed of the condition of the student. The teacher was able to appropriately document and log the behavior event and the student privileges were assigned accounting for his condition.

The apparatus and methods disclosed herein may be embodied in other specific forms without departing from their spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A system that interacts with one or more guardians of one or more students comprising:
a first electronic device including a processor and memory non-transitively programmed to:
receive identification information of the one or more students in relation to event information, location information, and/or behavior information of the one or more students;
a second electronic device including a processor and memory non-transitively programmed to:
receive the identification information related to the one or more students and the event information, the location information, and/or the behavior information;
determine, using the second electronic device, one or more actions to take based on the identification information, the event information, the location information and/or the behavior information of the one or more students; and
a third electronic device including a processor and memory non-transitively programmed to:
receive the one or more determined actions and interactively notify the one or more guardians of the one or more students of a final determined action.

2. The system of claim 1, wherein the third electronic device is a smart phone, tablet, or computer system of the one or more guardians.

3. The system of claim 2, wherein the third electronic device interactively notifies the one or more guardians by requiring the one or more guardians to acknowledge the final determined action or approve the final determined action.

4. The system of claim 1, wherein the first electronic device receives the identification information by scanning indicia on an identification card of the one or more students or by manual input of identification information of the one or more students.

5. The system of claim 3, wherein the final determined action is a notice that the one or more students have one or more of missing assignments, late assignments, quizzes, presentations, projects, important homework due, or all assignments are turned in.

6. The system of claim 1, wherein the third electronic device interactively notifies the one or more guardians by requiring the one or more guardians to approve a tardy or absent child and the final notification is of the approved tardy or absence.

7. The system of claim 1, wherein the indicia is a machine readable image, a barcode, or an alphanumeric code.

8. The system of claim 1, wherein the event is a financial charge to an account of the one or more students.

9. The system of claim 1, wherein the behavior is positive behavior or negative behavior.

10. The system of claim 1, wherein the location information is determined, in part, by GPS (global positioning system) within the first electronic device.

11. The system of claim 1, wherein the location information is determined by user input within the first device.

12. The system of claim 1, wherein a user of the first device is one or more of a teacher, an administrator, support staff, campus security, or a bus driver.

13. The system of claim 12, wherein the user of the first device is authenticated by scanning an identification card of the user.

14. The system of claim 1, wherein the second electronic device is a server, database server, private server, intranet server, remote computer, or cloud-based computing system.

15. The system of claim 1, wherein the third electronic device interactively notifies the one or more guardians by requiring the one or more guardians to choose from a selection of determined actions before the final determined action is presented to the one or more guardians.

16. The system of claim 1, wherein the third electronic device displays a historical log of locations to the one or more guardians of the one or more students.

17. The system of claim 16, wherein the historical log includes an indication of the one or more students being in a correct location at a correct time.

18. The system of claim 17, wherein the correct location is a classroom, lunchroom, bus, or home.

19. The system of claim 18, wherein one or more guardians can request a message to be delivered to the one or more students through interaction with the third electronic device.

20. The system of claim 19, wherein the message is delivered to the one or more students when the student identification card is scanned by the first electronic device after the request by the one or more guardians.
